(19) United States
(12) Patent Application Publication
(10) Pub. No.: US 2010/0056484 A1
(43) Pub. Date: Mar. 4, 2010

(54) DIETARY SUPPLEMENTAL COMPOSITION EFFECTIVE FOR ENHANCING COGNITIVE PERFORMANCE, ELEVATING MOOD AND REDUCING OXIDATIVE STRESS

(76) Inventor: Scott Farese, Vero Beach, FL (US)

Correspondence Address: MCHALE & SLAVIN, P.A., 2855 PGA BLVD, PALM BEACH GARDENS, FL, 33410, US

(21) Appl. No.: 12/198,622

(22) Filed: Aug. 26, 2008

(51) Int. Cl. A61K 31/661; A61P 25/00

(52) U.S. Cl. 514/114

(57) ABSTRACT

A dietary supplemental composition composed of Acetyl-L-Carnitine, Phosphatidylserine, L-Alpha-Glycerylphosphorylcholine, fish oils including Docosahexaenoic acid, Alpha Lipoic Acid, and optionally Eicosapentaenoic acid, and which in combination has been demonstrated to be effective in increasing cognitive performance, elevating mood and decreasing oxidative stress levels in mammals.

### FIELD OF THE INVENTION

This invention relates to a composition comprised of amino acids, phospholipids, fatty acids and fish oils, and anti-oxidants; and particularly to a dietary supplemental composition wherein the composition is administered daily to enhance cognitive performance, elevate mood and reduce oxidative stress.

### BACKGROUND OF THE INVENTION

Memory loss affects individuals at differing levels, degrees and at various periods in one's life. Almost all individuals have suffered from the inability to recall a person's name, place, or recent event. For most people, this type of memory loss is short lived, amounting to nothing more than temporary embarrassment or reinvestment of time to recall the forgotten item. Furthermore, modern society is complex often requiring individuals to recall large amounts of data. Present day society is much more technologically advanced than previous times, allowing individuals the ability to communicate and receive information almost instantly. Reliance on computers, smart-cell phones and wireless technology, has increased the need for memory recall and ability to process information quickly and clearly.

As individuals begin to age, a natural decline in ability to recall vast amounts of learned behavior stored over a lifetime develop. Parts of the brain responsible for memory can be vulnerable to age-related deterioration which affects a person's ability to recall information. Age-related loss of neurons affects the ability of the brain to communicate with cells that carry out cognitive functioning. This type of memory loss is a function of the brain weakening in its ability to store and retrieve new information rather than more serious pathological problem. As a result, we begin to have “normal” forgetfulness, which includes greater episodes of forgetting where you place certain objects, names of people, or an occasional forgetting of an appointment. Generally occurring around the ages of 50-60, it is not usually associated with having too much of a negative impact on the daily lives of such individuals.

At the other end of the spectrum are the types of memory loss associated with neurodegenerative diseases. Memory loss associated with these diseases affect a person's daily life making it difficult to work and function normally. Moreover, it is difficult to sustain various relationships putting a strain on family members. Although it may difficult to determine when normal memory loss is indicative of disease association, affecting a person's ability to remember routine activities could be associated with the more serious group of dementia related illnesses.

One type of dementia related illness, Alzheimer's disease (AD), is a progressive degenerative brain syndrome affecting memory, thinking and behavior and ability to express thoughts and emotions. This disease destroys brain cells and nerves, resulting in a decreased ability of the brain to carry messages regarding many normal functions. The disease is characterized by the formation of plaques. These structures are deposits of amyloid protein built up between nerve cells. Although the exact role plaques play in AD is currently unknown, the formation of these plaques is believed to be an important part of the pathology of the disease. As the population ages, cases of AD are expected to sharply rise with some predictions of 22 million cases in the U.S. by 2050. The current estimated medical related costs for those suffering AD can reach $50,000 annually, potentially straining, if not crippling, the health care system.

In the last several years there has been an increasing interest in the possible role of oxidative stress in AD. Several reports suggested increased lipid peroxidation in the temporal cortex, increased protein oxidation in the frontal cortex, and increased DNA oxidation in the parietal cortex of AD patients. Antioxidants are believed to be important in health maintenance through the modulation of oxidative processes in the body. Oxidative damage with the unregulated production of reactive oxygen species (ROS) such as hydrogen peroxide and hydroxyl radicals has been implicated in a growing number of clinical disorders such as atherosclerosis, stroke, Parkinson's disease and AD. Mechanisms responsible for the ROS-mediated injury to cells and tissues mainly include lipid peroxidation, oxidative DNA damage, and protein oxidation, but there is also evidence that ROS can induce the process of cell death. Indeed, unbalance in the endogenous antioxidant system can modulate cellular proliferation, either in a positive or a negative way, respectively leading to stimulation in cell proliferation at low levels of peroxides or to apoptotic/necrotic cell death at higher concentrations.

Given the range of memory loss an individual may suffer and the impact memory-related diseases have on the health care system, it is not surprising that many methods have been developed to avert memory loss. Preventing memory loss has created an entire industry developing approaches such as books, puzzles and pharmaceutical-type cures. Overcoming less severe memory loss can be associated with increasing brain function activities. With this method, individuals use various games, puzzles or intellectual techniques to keep the brain active, attempting to keep the brain functioning in a “use it or lose it” mentality. Various memory enhancing strategies include mentally associating new information with old information, placing items in the same place everyday, or creating written notebooks. Other ways to decrease memory loss includes eating certain foods and maintaining a healthy diet, limiting alcohol consumption, proper exercise and sleep, supplemental vitamins, and close social support with others. In fact, over the last 30 years, numerous pharmaceuticals and dietary supplements, such as vitamins, fish oils, phospholipids and various amino acids, have been researched in attempts to develop nutritional supplements to fight various diseases or human ailments, including enhancing mood and cognitive performance.

U.S. Pat. No. 6,214,378 discloses and claims a capsule formulation with DHA and EPA for oral preparation which is useful for colon diseases such as colon cancer, ulcerative colitis, constipation and diarrhea and for systemic diseases such as osteoporosis. Such capsules do not undergo any change in the stomach or small intestine. Disintegration begins upon arriving within the large intestine and, at the same time, quickly releases the drug. The capsule base is hydroxypropylmethylcellulose (HPMC) or polyethyleneglycol-compounded HPMC, gelatin or agar. On the surface of the capsule base is a powder or liquid containing a pharmacologically active substance, which is encapsulated and forms a double-coated structure comprising an inner layer consisting of a cationic copolymer and an outer layer consisting of anionic copolymer.

U.S. Patent Publication No. 2003/0165596 discloses a nutritional composition for use as a nutritional supplement to a diet on a regular basis, e.g. on a daily basis. The composition comprises vitamin compounds comprising a selection of vitamins, mineral compounds comprising a selection of minerals, and fish oil granulate in dry pulverized form comprising eicosapentaenoic acid (EPA) and docosahexaenoic acid (DHA).

U.S. Patent Publication No. 2004/0235795 discloses the use of phosphatidylserine (PS), lyso-phosphatidylserine or/and a physiologically acceptable salt for the treatment of the attention deficit syndrome (ADHS). In addition, additives selected from the group comprising antioxidants, essential fatty acids, mineral substances, amino acids, mood-lifters or/and phospholipids are added. Administration of the composition is to individuals aged 2 to 20 years, preferably 3 to 10 years, whereby phosphatidylserine as well as the additives are used in solid and also in liquid formulations.

U.S. Patent Application No. 2005/0171200 discloses a method for preventing cerebral damage in patients having symptoms of atherosclerosis of arteries supplying the brain by administering to the patient a therapeutically effective amount of a composition comprising eicosapentaenoic acid (EPA), docosahexaenoic acid (DHA) or a through a combination of the two.

U.S. Patent Application No. 2006/0166935 discloses the use of a fatty acid composition comprising at least (all-Z omega-3)-4,7,10,13,16,19-docosahexaenoic acid (DHA), or derivatives thereof, and (all-Z omega-3)-5,8,11,14,17-eicosapentaenoic acid (EPA), or derivatives thereof for manufacturing of a medicinal product or a food stuff for the treatment and/or prevention of amyloidos-related diseases, such as Alzheimer's disease, as well as treatment/prevention of cognitive dysfunction.

U.S. Patent Application No. 2006/0188607 discloses oral dosage compositions which contain edible oils, preferably containing an omega-3 fatty acid, in combination with one or more water soluble vitamins and/or minerals, such as vitamins B6, B9, and/or B12, beeswax and gelatin. The present invention also provides a method of making the composition comprising mixing the edible oil and one or more water-soluble vitamins and/or minerals to form a suspension or emulsion of the water-soluble vitamins and/or minerals in the edible oil. The mixture can be inserted into capsules, gel caps, or caplets for oral consumption. Additionally, the edible oil can coat particles of the water-soluble vitamins and/or minerals, which may preferably provide the vitamins and/or minerals improved absorption in the body due to increased resistance to degradation in the acidic environment of the stomach.

U.S. Patent Application No. 2007/0014866 discloses therapeutic compositions for reducing triglycerides, lowering LDL and increasing HDL. The composition is formed by combining either pantethine or CoA, or a combination of pantethine and CoA with fish oils. Either pantethine or CoA, or a combination of pantethine and CoA, added to cardiovascular drugs or compositions for lowering cholesterol increases the therapeutic effects and decreasing the side effects of those drugs or compositions. Either pantethine or CoA, or a combination of pantethine and CoA, added to drugs or compositions used in the treatment of Type I or Type II diabetes also increases the therapeutic effects and decreasing the side effects of those drugs or compositions.

U.S. Patent Application No. 2007/0037777 discloses anabolic compositions which can be administered to patients with chronic diseases, or who suffer from conditions precipitated by such diseases or long term treatment regimes. The compositions comprise at least one surfactant wherein the total concentration of surfactant in the composition is greater than 1% (w/w or w/v), and can cause weight gain in chronic disease patients as well as reduce or eliminate other disease symptoms or conditions. In particular, use of DHA, EPA and phosphatidyl for use in cancer treatment is disclosed. The anabolic compositions can also provide therapeutic benefit in patients who are candidates for, or who have undergone, organ or tissue transplant procedures.

Therefore, there is a need for a dietary supplemental compound having Acetyl-L-Carnitine, Phosphatidylserine, L-Alpha-Glycerylphosphorylcholine, Fish oils including eicosapentaenoic acid (EPA) and docosahexaenoic acid (DHA), and Alpha Lipoic Acid (ALA), which in specific combination provide for enhancement of cognitive performance, elevation in mood and reduction of oxidative stress in dietary deficient mammals.

### SUMMARY OF INVENTION

The present invention relates to several compounds, which in combination have been shown to increase cognitive performance and mood and reduce oxidative stress in dietary deficient mammals. It is contemplated that the mammal may be a human being. Among the compounds investigated were Acetyl-L-Carnitine (ALCAR), Phosphatidylserine, L-Alpha-Glycerylphosphorylcholine (Alpha-GPC), Fish oils including eicosapentaenoic acid (EPA) and docosahexaenoic acid (DHA), and Alpha Lipoic Acid (ALA). The combination of the components of the present invention has been registered under the trademark name of MEMACIN.

### Acetyl-L-Carnitine

Acetyl-L-Carnitine (ALCAR) is a naturally occurring acetyl ester of L-Carnitine. L-Carnitine functions the oxidation mechanism of fatty lipids, including transporting fatty acids across the mitochondrial membranes, allowing fatty acid metabolism to produce the necessary energy for the cellular functions. Functionally, ALCAR is a deliverable form of L-Carnitine and acetyl groups. ALCAR is an endogenous mitochondrial membrane compound that helps mitochondrial bioenergetics and lowers the increased stress associated with aging.

It exerts unique neuroprotective, neuromodulatory, and neurotrophic properties, which play an important role in counteracting various pathological processes, and has antioxidative properties, protecting cells against lipid peroxidation. Glutathione (GSH) is an important endogenous antioxidant, and its levels have been shown to decrease with aging. Administration of ALCAR increases cellular levels of GSH in rat astrocytes. ALCAR is derived from Carnitine and is described as having several properties which may be beneficial in dementia.

### Phosphatidylserine

Phosphatidylserine (PS) is a phospholipid, structurally consisting of a glycerophosphate backbone linked to fatty acids and the amino acid L-serine. Like other phospholipids, PS is a structural element of various plant and animal cells, particularly part of the cell membrane which keeps the cell intact and is responsible for moving nutrients in and out of the cell. In addition to being part of the basic structural element of biological membranes, PS is involved in the signal transduction pathway. Although found in all cell types, PS is particularly concentrated in the brain. Its role in nerve cell function is thought to involve transmitter release and synapse activity. In various studies, PS has been found to enhance mind and memory enhancement, including improvements in memory, learning, concentration, and recall in middle-aged men with dementia or age-related memory impairments. Research over the last 25 years indicated that PS maybe used to help improve the quality of life in patients suffering from Alzheimer's Disease and as well as improving memory and mental deterioration in aging non-Alzheimer's Disease suffering patients.

### L-Alpha-Glycerylphosphorylcholine

L-Alpha-Glycerylphosphorylcholine (Alpha-GPC) is a naturally occurring phospholipid precursor derived from soy lecithin. In several studies, Alpha-GPC has been shown to affect several neurotransmitter systems which may be involved in the development of age-related memory impairments. It is believed that Alpha-GPC may enhance cognitive function through the release of free choline which is then used by biosynthesis pathways in the brain. Other studies have shown that Alpha-GPC may increase inositol phosphate formation in the brain resulting in greater availability of phosphoinositides available for signal transduction.

### FISH OILS: Eicosapentaenoic Acid (EPA) and Docosahexaenoic Acid (DHA)

Fish oils are lipids commonly found in fatty fish, and have been studied because of their high concentrations of Omega 3-fatty acids. The two most commonly found Omega 3-Fatty acids are eicosapentaenoic acid (EPA) and docosahexaenoic acid (DHA). These two fish oils have been found to have several very important health related activities. Because of their triglyceride-lowering activity, scientists have studied these compounds for their role in lowering heart disease. However, EPA and DHA have several actions in various other body systems. In addition to the cholesterol lowering function, these two compounds have anti-inflammatory, anti-thrombotic, and immunodulatory actions.

EPA and DHA may also affect the human brain. DHA is vital to the developing fetus and infant brain and for normal brain function throughout a human's life. High levels of fish or fish oils in one's diet may be linked to decreased age-related memory loss and cognitive function impairment and lower risk of developing Alzheimer's disease.

### Alpha Lipoic Acid

Alpha Lipoic Acid (ALA) is a naturally occurring substance that is made in small amount by both plant and animal cells. Endogenously produced ALA is bound to specific proteins which function as cofactors for several mitochondrial enzyme complexes. Moreover, free existing, exogenously produced ALA has been shown to have antioxidant properties. In addition to these functions, ALA, in combination with L-Carnitine, has been found to improve memory related measurements of age-related cognitive decline in rats and mice.

Accordingly, it is a primary objective of the instant invention to provide a composition which in combination increases cognitive performance in mammals, and particularly in humans.

It is another objective of objective of the instant invention to provide a composition which in combination enhances mood in mammals.

It is a further objective of the instant invention to provide a composition that can improve cognitive performance in mammals despite dietary deficiencies and dietary oxidative stress.

It is yet another objective of the instant invention to provide a composition that can reduce oxidative stress.

Other objects and advantages of this invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### BRIEF DESCRIPTION OF THE FIGURES

FIG. 1 represents results of a Y-maze test indicating levels of increased cognitive performance of mice placed on a complete diet, complete supplemented with MEMACIN and mice on the deficient diet with or without supplementation with MEMCACIN.

FIG. 2 represents decreases in reactive oxygen species in the mouse brain as measured by TBAR levels, measuring differences in TBAR concentrations between mice in the complete diet complete category and complete diet plus MEMACIN category and between mice in the deficient diet category diet and deficient diet plus MEMACIN category.

### DETAILED DESCRIPTION OF THE INVENTION

### Scientific Data and Human Testimonials:

A new dietary supplement composition composed of Acetyl-L-Carnitine, Phosphatidylserine, L-Alpha-Glycerylphosphorylcholine, Fish oils including eicosapentaenoic acid (EPA) and docosahexaenoic acid (DHA), and Alpha Lipoic Acid (ALA), under the trademark name of MEMACIN, was administered to mice in order to determine the effectiveness of various dietary supplements in combination with regard to cognitive performance and levels of reactive oxygen species.

In addition to data collected using a mouse model system, human testers also received the dietary supplemental composition (MEMACIN) and reported by means of testimonials, after taking specific amounts of MEMACIN, particularly noting MEMACIN effect on mental sharpness, energy levels or other noticeable benefits.

The composition can be administered in any formulation, including but not limited to a liquid, gel, powder, pill, capsule, or gel-capsule:

### EXAMPLE 1

| Compound | Amounts |
|---|---|
| Acetyl-L Carnitine | up to about 300 mg |
| Phosphatidylserine | up to about 100 mg |
| L-Alpha-Glycerylphosphorylcholine | up to about 300 mg |
| Docosahexaenoic Acid | up to about 1200 mg |
| Eicosapentaenoic Acid | up to about 180 mg |
| Alpha Lipoic Acid | up to about 135 mg |

Other ingredients: Gelatin, glycerin, soybean oil, purified water and yellow beeswax

### EXAMPLE 2

| Compound | Amounts |
|---|---|
| Acetyl-L Carnitine | about 300 mg |
| Phosphatidylserine | about 100 mg |
| L-Alpha-Glycerylphosphorylcholine | about 300 mg |
| Docosahexaenoic Acid | about 400 mg |
| Eicosapentaenoic Acid | about 180 mg |
| Alpha Lipoic Acid | about 100 mg |

Other ingredients: Gelatin, glycerin, soybean oil, purified water and yellow beeswax

### EXAMPLE 3

| Compound | Amounts |
|---|---|
| Acetyl-L Carnitine | about 105 mg |
| Phosphatidylserine | about 54 mg |
| L-Alpha-Glycerylphosphorylcholine | about 210 mg |
| Docosahexaenoic Acid | about 1167 mg |
| Alpha Lipoic Acid | about 135 mg |

Other ingredients: Gelatin, glycerin, soybean oil, purified water and yellow beeswax

### Introduction and Rationale for Rat Model.

A growing body of research indicates that nutritional deficiencies contribute to age-related cognitive decline. For instance in neurodegenerative disease of Alzheimer's disease, increase of oxidative stress is one risk factor associated with age-related decline in cognitive performance. Key genetic and/or environmental factors may remain latent pending age-related decline in nutrition (Mattson and Shea, 2002). This suggests the potential importance of early nutritional intervention, including preventative approaches prior to definitive diagnosis (Burgener et al., 2008; Panza et al., 2004). Oxidative stress is a pivotal factor in AD, and is evident prior to cytopathological hallmarks of the disorder (Zhu et al., 2007). Antioxidants therefore represent a potential preventative approach (Joseph et al., 2000; Mattson and Shea, 2002).

Folate deprivation promotes age-related neurodegenerative disorders including Alzheimer's disease (AD; Mattson and Shea, 2002). Folate deprivation is potentiated by vitamin E deficiency (Shea and Rogers, 2002). We have also determined that inclusion of a high concentration of iron can further exacerbate folate and vitamin E deficiency. This has become our routine model for examining the efficacy of various dietary supplements.

Cognitive performance of normal mice is subject to dietary compromise (Mihalick et al., 2004). In addition, a major genetic risk factor for Alzheimer's disease is the presence of the E4 allele of apolipoprotein E (ApoE; Growdon, 2001; Rebeck et al., 2002). Oxidative damage in the brain is elevated in AD patients, and the extent of this damage correlates with the presence of the E4 allele (Ramassamy et al., 1999). Transgenic mice lacking Apo E (“ApoE−/− mice”) exhibit increased oxidative stress, and represent a useful model for the impact of oxidative stress on age-related neurodegeneration, which is accompanied by cognitive decline (e.g., Huang et al., 2000; Ramassamy et al.,1999, 2001; Shea and Rogers, 2002; Tchantchou et al., 2004a,b, 2005, 2006a,b). Key dietary deficiencies (such as maintenance on our deficient diet) can exacerbate this decline, while critical supplementation can minimize or prevent it. ApoE−/− mice exhibit increased susceptibility to folate deprivation: within 1 month of folate deprivation, ApoE−/− mice, but not normal mice, display oxidative damage in brain tissue in a gene-dose manner, impaired cognitive performance (Mihalick et al., 2004; Shea and Rogers, 2002; Shea et al., 2002a,b, 2004; Tchantchou et al., 2004a, 2005, 2006a,b). ApoE−/− mice therefore provide a useful index for Alzheimer's disease but, importantly, also encompass cognitive decline during normal aging. Coupled with normal adult mice, they further provide a model for the deleterious impact of nutritional, environmental and genetic risk factors on cognitive decline.

### Methodology

### Mice and Diet

A total of 36 adult, normal C57B/6 mice, ages 9-12 months were used in this study. Mice were grouped in sets of three and placed in four groups depending on diet received with or without receiving the dietary supplement: complete diet, deficient diet, complete diet plus MEMACIN, and deficient diet plus MEMACIN. In addition to the diet, Mice in each group were given water ad libitum and maintained this regimen for one month. Several mice received a diet (“AIN-76”; Purina/Mother Hubbard, Inc.) lacking folate and vitamin E which was defined as the “deficient diet.” The deficient diet was also supplemented in all cases with iron (50 g/500 g total diet) as a pro-oxidant (Mihalick et al., 2004; Shea and Rogers, 2002; Tchantchou et al., 2004a). Other mice received a diet supplemented with folic acid (4 mg/kg), and vitamin E (50 IU/kg total diet wet weight) and was defined as the “complete diet.” Additional groups of mice maintained on either the complete diet or deficient diet further received the MEMACIN for a one month period at a various compositions as described by Examples 1-3. Mice on the complete diet with MEMACIN supplements were grouped as “complete plus MEMACIN.” Mice on the deficient diet but supplemented with MEMACIN were labeled as “deficient plus MEMACIN.” This regimen was completed 3 times, for a total of 9 mice per diet.

### Assessment of Cognitive Behavior: Y-Maze Test

Cognitive performance was assessed through subjecting the animals to a standard Y-maze tests as described Mihalick et al. (2004) Folate and vitamin E deficiency impair cognitive performance in mice subjected to oxidative stress: differential impact on normal mice and mice lacking apolipoprotein (E. Neuromol Med 4:197-202), the contents of which are herein incorporated by reference. Briefly, before and after the feeding regimen as outlined above, mice were placed in a device having three corridors, or arms shaped in a Y and analyzed for the choices of arms entered and frequency of visitation. In general, mice have a tendency to explore all three arms in the maze in succession which is generally viewed as a successful performance. The pattern of exploration of the Y maze was recorded over 5 min intervals and the percent alternations were determined for each animal. The percent alternations were defined as the frequency in which mice visited each of the 3 arms during any 3-arm visitation sequence.

### Analysis of Oxidative Damage to the Brain: TBAR Tests

Following the final Y maze test, mice were sacrificed by cervical dislocation, and the frontal portion of the brain (encompassing cortex and hippocampus) and the liver were immediately removed and frozen for subsequent analyses. An increase in oxidative damage is known to accompany maintenance on the deficient diet (e.g. Shea and Rogers, 2002). In these analyses, liver provides an index of total body reactive oxygen species, which can indicate any specificity of brain protection. In addition to the TBAR assay method as described by Shea T B and Rogers E (2002). Folate quenches oxidative damage in brains of apolipoprotein E-deficient mice: augmentation by vitamin E. Mol Brain Res 108:1-6, the contents of which are herein incorporated by reference, use of commercial assay kits to perform analysis on multiple samples at one time may be used.

### Results

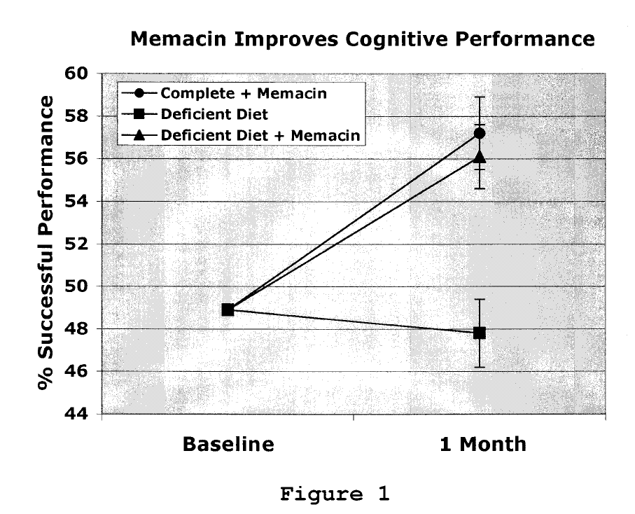

Results of the Y-maze test indicate improvement of general cognitive functioning. As seen in FIG. 1, alternation frequencies were observed for mice on the deficient diet, complete diet plus MEMACIN and deficient diet plus MEMACIN. A 17% improvement in cognitive performance of normal mice following 1 month maintenance on the complete diet supplemented with MEMACIN was observed. Additionally, a 15% improvement for mice maintained on the deficient diet supplemented with MEMACIN was observed. No alteration of cognitive performance was observed for the deficient diet alone.

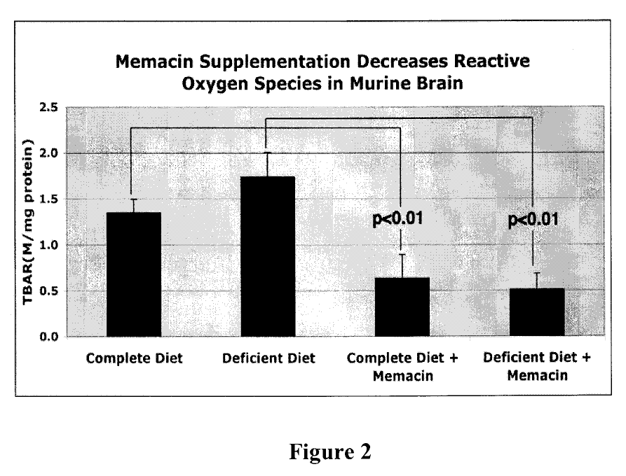

In addition, levels of brain reactive oxygen species as measured by Thiobarbituric Acid Reactive Substances (TBAR) levels for mice in all groups were determined. FIG. 2 shows the results of TBAR levels in the mouse brain, comparing levels between mice in the complete diet group with mice in the complete diet plus MEMACIN group and comparing levels of mice in the deficient diet group with mice in the deficient diet plus MEMACIN group. Brain homogenates from each of the four groups, complete diet, deficient diet, complete diet plus MEMACIN and deficient diet plus MEMACIN were processed and examined for levels of TBAR. As seen in FIG. 2, levels of TBAR decreased in comparing complete diet group in when compared to the mice in the complete diet plus MEMACIN. Furthermore, levels of TBAR decreased in comparing mice in the deficient diet with mice in the deficient diet plus MEMACIN. These results indicate that, in addition to improving cognitive behavior, MEMACIN also reduces oxidative damage to the brain tissue of mice maintained on both complete and deficient diets.

The results of the Y-maze test and measurements of TBAR levels, taken together, indicate that MEMACIN can improve cognitive performance despite dietary deficiency and dietary oxidative stress.

In addition to testing in mice, human testers ranging in age and gender were given sample trials of the MEMACIN supplements for periods of time ranging from 1-2 weeks, 2-4 weeks, and 4 or more weeks. Individuals were given MEMACIN pills in concentrations as exemplified by Examples 1-3. Pills were taken 1-3 times daily. After completing their trial period, individuals reported results of the trials. Particularly, individuals reported on mental sharpness, energy levels and any benefits observed while taking the pills.

In evaluating mental sharpness and energy levels, patients were asked to rate the any changes through a rating system. Of the testers that responded, 73% reported increases in mental sharpness while taking MEMACIN. Moreover, within this group, 73% reported mental sharpness increases at the highest level of the rating scale. With regard to evaluation of energy levels, 80% reported increase in energy levels while taking MEMACIN. Within this group, 83% reported the energy level increases at the highest rating of the rating system.

### Human Testimonial 1:

Male individual taking 2 pills of MEMACIN daily for a period of 2 weeks, reported, at the end of the trial, highest rating of increased mental sharpness and energy levels.

### Human Testimonial 2:

Female individual taking 2 pills of MEMACIN daily reported the highest rating of increases in mental sharpness and energy levels.

### Human Testimonial 3:

Female individual taking 3 pills of MEMACIN for a 2-4 week period reported highest level of rating regarding increase in mental sharpness levels and increase in energy levels.

In a particularly preferred, albeit non-limiting embodiment, the composition of the invention which is effective for enhancing cognitive performance and elevating mood is supplied in unit ratios of about 3 parts by weight Acetyl-L-Carnitine, about I part by weight Phosphatidylserine, about 3 parts by weight L-Alpha-Glycerylphosphorylcholine, about 3 parts by weight Docosahexaenoic acid, and about 1 part by weight Alpha Lipoic Acid. The composition may further include from about 1 part to about 3 parts by weight (5 mg to about 600 mg) of Eicosapentaenoic acid.

The composition is provided in a unit dosage wherein each part by weight is in the range of about 5 mg to about 200 mg, therefore when each tablet or capsule is provided in the contemplated unit dosage ratio, the total weight per capsule will be in the range of from about 75 mg:25 mg to about 225 mg:75 mg. In a preferred embodiment, a daily dosage would be three 100 mg tablets, gel caps or the like, each containing a 75 mg:25 mg ratio of the five ingredients. In some instances, it may be preferable to increase the dosage of Docosahexaenoic Acid to about 1200 mg.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A composition effective for enhancing cognitive performance and elevating mood when administered to mammals, comprising in combination about 3 parts by weight Acetyl-L-Carnitine, about 1 part by weight Phosphatidylserine, about 3 parts by weight L-Alpha-Glycerylphosphorylcholine, about 3 parts by weight Docosahexaenoic acid, and about 1 part by weight Alpha Lipoic Acid.

2. The composition of claim 1, further containing from about 1 part to about 3 parts by weight of Eicosapentaenoic acid.

3 The composition of claim 1 wherein each part by weight is in the range of about 5 mg to about 200 mg.

4. The composition of claim 1 wherein each tablet or capsule is in a unit dosage ration in the range of from about 75 mg:25 mg to about 225 mg:75 mg.

5. The composition of claim 1 wherein a recommended daily dosage is from about 15 mg-600 mg Acetyl-L-Carnitine, from about 5 mg-200 mg Phosphatidyl Serine; from about 15 mg-600 mg L-Alpha-Glycerylphosphorylcholine, from about 15 mg-600 mg Docosahexaenoic Acid, and from about 5 mg-200 mg Alpha Lipoic Acid.

6. The composition of claim 5 wherein the recommended daily dosage of Docosahexaenoic Acid is about 1200 mg.

7. The composition of claim 5, further including about 5 mg to 600 mg Eicosapentaenoic Acid.

8. A composition effective for enhancing cognitive performance and elevating mood when administered to mammals at the rate of Acetyl-L-Carnitine, about 300 mg; Phosphatidyl Serine, about 100 mg; L-Alpha-Glycerylphosphorylcholine about 300 mg; Docosahexaenoic Acid, about 400 mg; Eicosapentaenoic Acid, about 180 mg; and Alpha Lipoic Acid, about 100 mg.

9. A composition effective for enhancing cognitive performance and elevating mood when administered to mammals at the rate of Acetyl-L-Carnitine, about 105 mg; Phosphatidyl Serine, about 54 mg; L-Alpha-Glycerylphosphorylcholine about 210 mg; Docosahexaenoic Acid, about 1167 mg; and Alpha Lipoic Acid, about 35 mg.
